(19) United States
(12) Patent Application Publication
(10) Pub. No.: US 2007/0287876 A1
(43) Pub. Date: Dec. 13, 2007

(54) Method of removing organic acid from light fischer-tropsch liquid

(76) Inventors: Ghasem Pajoumand, Barrington, IL (US); Gregory J. Gajda, Mount Prospect, IL (US); Peter Kokayeff, Naperville, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/005,808

(22) Filed: Dec. 7, 2004

(51) Int. Cl. C07C 5/00; C07C 7/00

(52) U.S. Cl. 585/860; 585/254

(57) ABSTRACT

A method of removing an organic acid from Light Fischer-Tropsch Liquid (LFTL) comprising treating the LFTL with about 30 to 75% ammonium hydroxide, whereupon the organic acid is converted to an ammonium salt; and a method of converting an olefin in LFTL to a paraffin comprising contacting LFTL, which has been treated with about 30 to 75% ammonium hydroxide so as to convert organic acid in the LFTL to ammonium salt, with a cobalt/molybdenum catalyst in the presence of hydrogen in a fixed-bed reactor.

### TECHNICAL FIELD OF THE INVENTION

The present invention relates to a method of removing organic acid from Light Fischer-Tropsch Liquid (LFTL) and a method of converting an olefin in LFTL to a paraffin.

### BACKGROUND OF THE INVENTION

At times it is necessary to remove olefins from hydrocarbon streams due to their high reactivity. LFTL can contain as much as 40 wt-% olefins, up to 2 wt-% organic acids, and 2 to 3 wt-% alcohols. In order to use LFTL as a diesel-blending agent, more than 98% of the olefins need to be converted to paraffins. Unfortunately, LFTL can not be hydrotreated in the presence of a cobalt/molybdenum (Co/Mo) catalyst because the organic acids deactivate the metal function of the catalyst, thereby rendering it ineffective.

The present invention seeks to provide a method of removing the organic acids from LFTL so that the Co/Mo catalyst can be used in hydrotreating the LFTL. This other objects and advantages, as well as additional inventive features, will become apparent from the detailed description provided herein.

### BRIEF SUMMARY OF THE INVENTION

The present invention provides a method of removing an organic acid from LFTL. The method comprises treating the LFTL with about 30 to 75% ammonium hydroxide, whereupon the organic acid is converted to an ammonium salt.

The present invention further provides a method of converting an olefin in LFTL to a paraffin. The method comprises contacting LFTL, which has been treated with about 30 to 75% ammonium hydroxide so as to convert organic acid in the LFTL to ammonium salt, with a cobalt/molybdenum (Co/Mo) catalyst in the presence of hydrogen in a fixed-bed reactor.

### DETAILED DESCRIPTION OF THE INVENTION

The present invention is predicated, in part, on the discovery that water, while customarily used to neutralize feed acidity, is ineffective in removing organic acids from LFTL. In view of the foregoing, it has been discovered that organic acids can be removed from LFTL by treating the LFTL with ammonium hydroxide.

Therefore, the present invention provides a method of removing an acid, in particular an organic acid, from LFTL. The method comprises treating, otherwise referred to as washing, the LFTL with ammonium hydroxide, which converts the organic acid to an ammonium salt.

Any suitable source of ammonium hydroxide can be used. Ammonium hydroxide is available from numerous commercial suppliers including, for example, Sigma Chemical Co., St. Louis, Mo. Preferably, about 30 to 75%, more preferably about 40 to 75%, and most preferably about 50 to 75% ammonium hydroxide (as a solution molality) is used. The use of 50 to 75% ammonium hydroxide can result in removal of around 99% or more of the organic acid in the LFTL.

The present invention further provides a method of converting an olefin in LFTL to a paraffin. The method comprises contacting LFTL, which has been treated with about 30 to 75% ammonium hydroxide so as to convert organic acid in the LFTL to ammonium salt, with a Co/Mo catalyst in a fixed-bed reactor in the presence of hydrogen. Conditions at the inlet of the fixed-bed reactor can include a reactor pressure of about 1931 to 3999 kPa (280 to 580 psig) such as about 1931 to 3310 kPa (280 to 480 psig) or about 1931 to 2620 kPa (280 to 380 psig), a temperature of about 260° to 454° C. (500° to 850° F.) such as about 260° to 385° C. (500° to 725° F.) or about 260° to 316° C. (500° to 600° F.), a liquid hourly space velocity (LHSV) of about 3 to 6 such as about 4 to 6 or about 5 to 6, and about 10.4 to 20.8 standard cubic meters (400 to 800 standard cubic feet) such as about 10.4 to 18.4 standard cubic meters (400 to 650 standard cubic feet) or about 10.4 to 14.2 standard cubic meters (400 to 500 standard cubic feet) of hydrogen per barrel of LFTL processed.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

What is claimed is:

1. A method of removing an organic acid from Light Fischer-Tropsch Liquid (LFTL), which method comprises:
treating the LFTL with about 30 to 75% ammonium hydroxide, whereupon the organic acid is converted to an ammonium salt; and
removing the ammonium salt from the LFTL, whereupon the organic acid is removed from the LFTL.

2. A method of converting an olefin in LFTL to a paraffin, which method comprises contacting LFTL, which has been treated with about 30 to 75% ammonium hydroxide so as to convert organic acid in the LFTL to ammonium salt, with a cobalt/molybdenum (Co/Mo) catalyst in the presence of hydrogen in a fixed-bed reactor, which comprises an inlet and an outlet, whereupon the olefin is converted to a paraffin.

3. The method of claim 2, wherein the conditions at the inlet of the fixed-bed reactor include a reactor pressure of about 1931 to 3999 kPa (280 to 580 psig), a temperature of about 260° to 454° C. (500° to 850° F.), a liquid hourly space velocity of about 3 to 6, and about 10.4 to 20.8 standard cubic meters (400 to 800 standard cubic feet) of hydrogen per barrel of LFTL processed.
